(19) United States
(12) Patent Application Publication
(10) Pub. No.: US 2010/0268244 A1
(43) Pub. Date: Oct. 21, 2010

(54) STENT INTRODUCER APPARATUS

(75) Inventors: Palle Munk Hansen, Bjaeverskov (DK); Anders Ginge Jensen, Ringsted (DK)

Correspondence Address: COOK GROUP PATENT OFFICE, P.O. BOX 2269, BLOOMINGTON, IN, 47402, US

(73) Assignees: William Cook Europe ApS, Bjaeverskov (DK); Cook Incorporated, Bloomington, IN (US)

(21) Appl. No.: 12/652,940

(22) Filed: Jan. 6, 2010

(30) Foreign Application Priority Data
Apr. 16, 2009 (GB) 0906580.6

(51) Int. Cl. A61F 11/00; A61M 29/00

(52) U.S. Cl. 606/108; 606/191

(57) ABSTRACT

A dilator element 30 of a stent introducer apparatus 10 comprises a distal part 36 of soft, flexible material and a proximal part 38 of relatively hard and/or stiff material with a proximal end portion 35 which is tapered.

### CROSS-REFERENCE TO RELATED APPLICATIONS

This application claims priority of Great Britain Application No. 0906580.6, filed Apr. 16, 2009.

### TECHNICAL FIELD

The present invention is related to apparatus for introducing a stent member into a vessel of a patient's body.

### BACKGROUND OF THE INVENTION

After using a stent introducer apparatus to deploy a stent member at a desired location, it is then necessary to withdraw the introducer apparatus from the patient's body. A problem which has arisen in practice concerns the dilator or nose cone element which is located at the distal end of the introducer apparatus. During withdrawal inside the stent member and the introducer catheter there is a tendency for the proximal end of the dilator to catch or snag on the end of the stent member and/or the end of the introducer catheter. This is disadvantageous in that the stent member may be dislodged while trying to free the dilator, and it may not be possible to release the dilator at all.

With a view to overcoming this problem, US2004/0230286 proposes tapering the proximal surface of a dilator. This is stated to reduce the likelihood of an edge catching the stent during withdrawal and also guides movement of the end of an introducer catheter over the dilator element. Because, during introduction, the dilator element comes into contact with the interior of the patient's body vessels, it is generally made of a relatively pliant and soft elastomeric material. This has the disadvantage that the proximal tapering surface of the dilator element is also of relatively soft material, so that there is still a danger of the end of the stent member or the catheter becoming caught thereby e.g. by producing a depression in the soft material of the taper.

### SUMMARY OF THE INVENTION

Aspects of the present invention seek to overcome or reduce the above problem.

According to the present invention there is provided a dilator element comprising a body part of a first material and a proximal end part of a second material, said second material having a stiffness and/or hardness greater than that of said first material, and at least a proximal end portion of said proximal end part reducing in cross-section.

In the present specification, the term proximal is used for a part of the introducer apparatus which is nearer to an operator of the apparatus. The term distal is used for a part of the introducer apparatus which is further from an operator; thus the dilator or nose cone element is located at the distal end of the introducer apparatus.

The proximal end portion of the dilator element is preferably in the form of a conical taper.

The dilator element preferably comprises a distal end portion of relatively soft, flexible or pliant material and, fixedly attached thereto, a proximal end portion of relatively hard, stiff or rigid material. The materials may be respectively soft and hard nylon material.

The surface of the distal end is preferably smooth.

### BRIEF DESCRIPTION OF THE DRAWING

Preferred embodiments of the present invention will now be described, by way of example only, with reference to the accompanying drawings, of which:

FIG. 1 shows a stent member and an introducer apparatus therefor;

FIG. 2 shows an enlarged sectional side view of a dilator element of the introducer apparatus of FIG. 1 with preferred dimensions indicated in millimetres; and

FIG. 3 shows a sectional top view of the dilator element of FIG. 2.

### DETAILED DESCRIPTION

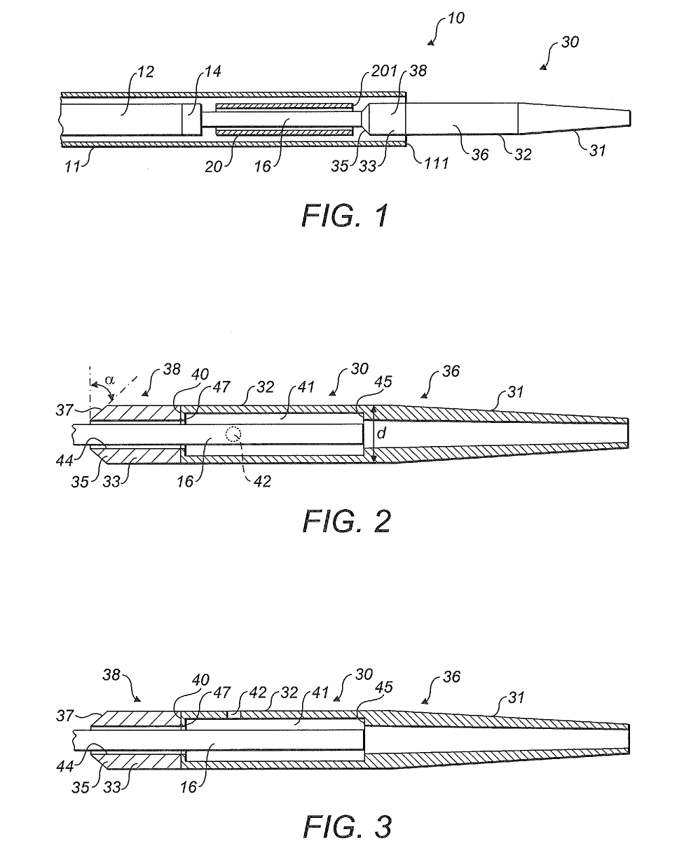

Referring to the drawings, FIG. 1 shows an introducer apparatus 10 for a stent member 20, such as a stent graft, the apparatus comprising an introducer catheter 11 surrounding a pusher assembly including an inner catheter 12 and a pusher ring 14. Within catheter 12 there is a guide wire catheter 16 having a nose cone or dilator element 30 at a distal end thereof. The dilator element 30 comprises a distal or body part 36 and a proximal part 38.

During a deployment procedure, the nose cone 30 is advanced through a body vessel of a patient and so, to reduce trauma to the patient the distal end portion 31 of part 36 is tapered, in particular the portion is of frusto-conical shape. To further reduce trauma the distal end part 36 is made of relatively soft and flexible nylon material. The end portion 31 is integrally formed with a central generally cylindrical portion 32.

Part 38 is fixedly attached to the proximal end of part 36 and is of relatively hard nylon material. It may have a Shore hardness of D50 to D65, preferably D58. Part 38 comprises a cylindrical portion 33 and an end portion 35 of frusto-conical shape and having a smooth surface 37.

Preferred dimensions of the dilator elements are:

| | |
|---|---|
| Maximum diameter d: | 1.33-2 mm (4-6 French) |
| Length of part 38: | 3 mm ± 1 mm |
| Length of cylindrical portion 32: | 7 mm ± 1 mm |
| Total length of dilator element 30: | 18 mm ± 2 mm |

FIGS. 2 and 3 respectively show sectional side and top views of dilator element 30, with parts 36 and 38 being connected together at an interface 40. Extending along most of the interior of the length of cylindrical portion 32 is a cylindrical cavity 41. The axial ends of the cavity are formed by a shoulder 45 and a flange 47. As shown in FIGS. 2 and 3 the right hand face of flange 47 defines the left hand end of the cavity and the left hand face defines the interface 40.

When making the dilator element 30, parts 36 and 38 are axially bonded together around a stepped mandrel. The internal diameter of the abutting ends of parts 36 and 38 initially have the same internal diameter. During the bonding process, applied pressure causes the material to flow to meet a smaller diameter portion of the mandrel, during which process flange 47 is formed.

Guide wire catheter 16 passes through a central bore 44 of the dilator element 30 and through the centre of cavity 41. A glue hole 42 communicates is between the cavity 41 and the exterior of part 36. During assembly of the dilator element 30 and the guide wire catheter 16, the latter is inserted into bore 44 as far as shoulder 45. The catheter fits snugly within the bore 44 within the proximal part 38. Glue is then injected through hole 42 to substantially fill cavity 41. The glue is allowed to set so that the dilator element 30 is firmly and permanently attached to catheter 16.

Turning now to use of the apparatus, as part of the deployment procedure of the stent member 20, one step is the withdrawal of dilator element 30. As the element 30 is withdrawn it is possible for it to come into contact with the end 111 of the introducer catheter or the end 201 of stent member 20. During the withdrawal operation, tapering portion 35 is effectively the leading end of the dilator element. If there is any contact between tapering portion 35 and end 111 or 201, the tapering portion is readily deflected away from the end and is free to continue being withdrawn (i.e. to the left in the Figure). The smooth nature of surface 37 ensures that the friction arising during the engagement of the tapering portion 35 with either of the ends 111, 201 is kept to a low value.

Thus an advantage of the stiff and hard nature of the end part 38 and in particular tapered portion 35 is that catching of the dilator element 30 on the stent member 20 or other parts of the introducer apparatus, in particular introducer catheter 11, is substantially prevented. Kinking of the dilator element is also avoided.

The use of embodiments of the invention in association with relatively long stent members is particular advantageous. The part 38 is relatively easy to produce, since hard materials are easier to shape than soft materials. The o connecting together of parts 36 and 38 is a simple and relatively quick procedure. The provision of a cylindrical portion 33 to the end part 38 means that less strain is put on the plane of connection to the distal part 36 and also that there is a larger surface area of stiff/hard material for deflection purposes. The assembly of the dilator element 30 and the catheter 16 is also a relatively is quick and easy procedure.

The parts 36, 38 may be connected by adhesion, thermal bonding or any other convenient method. If desired, cylindrical portion 33 may be omitted, so that tapered portion 35 is connected directly to the cylindrical portion 32 of distal part 36. To reduce the risk of kinking of the dilator element, the length of proximal part 38 is substantially shorter than the length of distal part 36. The length ratio lies between 1:3 and 1:8, preferably 1:4 to 1:6 and most preferably 1:5.

The size and positioning of glue hole 42 may be selected as desired; more than one glue hole may be provided.

Any suitable combination of materials may be used for parts 36 and 38. Elastomeric materials are suitable for part 36. Hard plastics materials are suitable for part 38; metals such as stainless steel may be used, but with an increased risk of kinking.

Instead of tapering frusto-conically, the diameter of portion 35 may reduce in other ways; for example it may have a part-spherical or other arcuate shape.

As shown the angle of the taper α is 45°. A range of angles between 40° and 60° can be employed, preferably between 45° and 60°.

1. A dilator element comprising a body part of a first material and a proximal end part of a second material, said second material having a stiffness and/or a hardness greater than that of said first material, and at least a proximal end portion of said proximal end part reducing in cross-section away from said body part.

2. A dilator element according to claim 1 wherein the said reduction in cross-section comprises a frusto-conical shape.

3. A dilator element according to claim 2, wherein the part with the frusto-conical shape has a smooth surface.

4. A dilator element according to claim 1 wherein a proximal end of the body part is fixedly attached to a distal end of the proximal end part.

5. A dilator element according to claim 1 wherein the proximal end part comprises a cylindrical body portion integral with a proximal end portion.

6. A dilator element according to claim 1 wherein the proximal end part reduces in cross-section along its entire length.

7. A dilator element according to claim 1 wherein the body part comprises a cylindrical body portion integral with a tapered distal end portion.

8. A dilator element according to claim 1 wherein the body part is of soft nylon material and the proximal end part is of hard nylon material.

9. A dilator element according to claim 1 wherein the ratio of the length of the body part to the length of the proximal end part lies within the range 6:1 to 4:1.

10. A dilator element according to claim 9 wherein said ratio is substantially 5:1.

11. A stent introducer apparatus comprising an introducer catheter surrounding a pusher assembly, said pusher assembly comprising an inner catheter and a pusher member, and a guide wire catheter arranged to carry a stent member to be deployed and having at a distal end thereof a dilator element comprising a body part of a first material and a proximal end part of a second material, said second material having a stiffness and/or a hardness greater than that of said first material, and at least a proximal end portion of said proximal end part reducing in cross-section away from said body part.

12. A stent introducer apparatus according to claim 11 wherein the dilator element is attached to the guide wire catheter by glue.
